(19) United States
(12) Patent Application Publication
(10) Pub. No.: US 2025/0281176 A1
(43) Pub. Date: Sep. 11, 2025

(54) HAND-HELD SURGICAL INSTRUMENTS

(71) Applicant: Covidien LP, Mansfield, MA (US)

(72) Inventors: David A. Nicholas, North Haven, CT (US); Russell Pribanic, North Haven, CT (US); David A. Zeichner, North Haven, CT (US); Robert H. Knapp, North Haven, CT (US)

(73) Assignee: Covidien LP, Mansfield, MA (US)

(21) Appl. No.: 19/088,005

(22) Filed: Mar. 24, 2025

Related U.S. Application Data
(63) Continuation of application No. 18/449,938, filed on Aug. 15, 2023, now Pat. No. 12,268,387.
Application No. 18/449,938 is a continuation of application No. 17/088,769, filed on Nov. 4, 2020, now Pat. No. 11,737,747.
(60) Provisional application No. 62/948,870, filed on Dec. 17, 2019.

(51) Int. Cl. A61B 17/068; A61B 17/00

(52) U.S. Cl. CPC A61B 17/0686; A61B 2017/00407

(57) ABSTRACT

A hand-held surgical instrument includes a handle housing, a shaft portion extending distally from the handle housing, and an articulation shaft. The articulation shaft may be configured to axially move within the shaft portion to articulate an end effector via a manual activation or a motor activation.

### CROSS-REFERENCE TO RELATED APPLICATION

This application is a continuation of U.S. patent application Ser. No. 18/449,938 filed on Aug. 15, 2023, which is a continuation of U.S. patent application Ser. No. 17/088,769 filed on Nov. 4, 2020, now U.S. Pat. No. 11,737,747, which claims the benefit of and priority to U.S. Provisional Application No. 62/948,870 filed on Dec. 17, 2019, the entire contents of which are incorporated by reference herein.

### BACKGROUND

### 1. Technical Field

The disclosure relates to surgical instruments. More specifically, the disclosure relates to hand-held electromechanical surgical instruments that articulate, rotate, and actuate a variety of other functions of surgical attachments, such as, for example, end effectors configured to staple tissue.

### 2. Background of Related Art

Electromechanical surgical instruments include a reusable handle assembly and disposable loading units and/or single use loading units, such as, for example, surgical end effectors. The end effectors are selectively connected to the handle assembly prior to use and then disconnected from the handle assembly following use in order to be disposed of or in some instances sterilized for re-use. Some surgical instruments may be capable of articulating the end effector to adjust the angle of orientation of the end effector during a surgical procedure. There are one or more drive mechanisms within the handle assembly for carrying out the articulation of the end effector and/or the operational functions of the end effector.

### SUMMARY

In one aspect of the disclosure, a hand-held surgical instrument is provided and includes a handle housing, a shaft portion extending distally relative to the handle housing, a first motor disposed within the handle housing, a ball screw operably coupled to the first motor, a ball nut non-rotationally supported in the shaft portion and operably coupled to the ball screw, and a firing shaft. The firing shaft has a proximal end portion attached to the ball nut, and a distal end portion configured to fire staples from an end effector. The ball nut is configured to translate the firing shaft along a longitudinal axis defined by the shaft portion in response to a rotation of the ball screw.

In aspects, the hand-held surgical instrument may further include a battery configured to power the first motor. In aspects, the battery may be configured to power all motors, LED's, and various other electronics.

In some aspects, the handle housing may have a barrel portion, and a handle portion extending perpendicularly from the barrel portion. The battery may be supported in the handle portion. In aspects, the battery may be supported in the barrel portion.

In other aspects, the handle portion may include an upper segment fixed to the barrel portion, and a lower segment pivotably coupled to the upper segment, the battery being disposed in the lower segment.

In further aspects, the handle portion may define a plane that extends parallel with the longitudinal axis of the shaft portion. The lower segment may be configured to pivot relative to the upper segment about a pivot axis that is parallel with the plane.

In aspects, the hand-held surgical instrument may further include a printed circuit board supported in the upper segment and configured to be in electrical communication with the battery and the first motor. In aspects, the printed circuit board may be in electrical communication with motion control circuitry.

In some aspects, the hand-held surgical instrument may further include a finger switch pivotably coupled to the upper segment. The finger switch may have an upper button and a lower button each in communication with the printed circuit board for activating the battery. In aspects, the finger switch may activate the battery and control the first motor.

In further aspects, the hand-held surgical instrument may further include a knob housing coupled to the handle housing, an articulation lever, and a first articulation shaft. The shaft portion may extend distally from the knob housing. The articulation lever may be rotationally coupled to the knob housing. The first articulation shaft may be operably coupled to the articulation lever, such that a rotation of the articulation lever translates the first articulation shaft to articulate an end effector.

In other aspects, the hand-held surgical instrument may further include a cam plate coupling the articulation lever with a proximal end portion of the first articulation shaft. The cam plate may be configured to urge the first articulation shaft in one of a proximal or distal direction upon rotation of the cam plate.

In aspects, the hand-held surgical instrument may further include an articulation locking assembly that includes a first ratchet gear operably coupled to the cam plate and a pawl engaged with the first ratchet gear. The pawl may be configured to restrict the rotation of the cam plate.

In some aspects, the first ratchet gear may be non-rotationally coupled to the articulation lever and pinned to the cam plate, such that a rotation of the articulation lever rotates the cam plate.

In further aspects, the cam plate may have a pin that extends through an elongate slot defined in the first ratchet gear. The first ratchet gear may be configured to rotate the cam plate after a delay.

In other aspects, the articulation locking assembly may include a second ratchet gear disposed between the first ratchet gear and the cam plate. The pawl may be engaged with the first and second ratchet gears.

In aspects, the first ratchet gear may have a plurality of teeth each defining an oblique surface, and the second ratchet gear may have a plurality of teeth each defining a linear surface.

In some aspects, adjacent teeth of the plurality of teeth of the first ratchet gear may define a triangular space therebetween, and adjacent teeth of the plurality of teeth of the second ratchet gear may define a rectangular space therebetween.

In further aspects, the second ratchet gear may be fixed to the cam plate, such that the cam plate and the second ratchet gear rotate simultaneously with one another.

In other aspects, the cam plate may define a first spiral slot, and the proximal end portion of the first articulation shaft may have a protuberance received in the first spiral slot.

In aspects, the hand-held surgical instrument may further include a second articulation shaft having a protuberance extending from a proximal end portion thereof. The protuberance of the second articulation shaft may be received in a second spiral slot defined in the cam plate. The first and second articulation shafts may be configured to translate in opposite directions in response to a rotation of the cam plate.

In accordance with another aspect of the disclosure, a hand-held surgical instrument is provided and includes a handle housing, a knob housing coupled to the handle housing, a shaft portion extending distally from the knob housing, an articulation lever rotationally coupled to the knob housing, a first articulation shaft, a cam plate, and an articulation locking assembly. The first articulation shaft is operably coupled to the articulation lever, such that a rotation of the articulation lever translates the first articulation shaft to articulate an end effector. The cam plate couples the articulation lever with a proximal end portion of the first articulation shaft. The cam plate is configured to urge the first articulation shaft in one of a proximal or distal direction. The articulation locking assembly includes a first ratchet gear operably coupled to the cam plate, and a pawl engaged with the first ratchet gear. The pawl may be configured to restrict the rotation of the cam plate.

In some aspects, the cam plate may have a pin that extends through an elongate slot defined in the first ratchet gear. The first ratchet gear may be configured to rotate the cam plate after a delay.

In further aspects, the articulation locking assembly may include a second ratchet gear disposed between the first ratchet gear and the cam plate. The pawl may be engaged with the first and second ratchet gears.

In other aspects, the first ratchet gear may have a plurality of teeth each defining an oblique surface, and the second ratchet gear may have a plurality of teeth each defining a linear surface.

In aspects, adjacent teeth of the plurality of teeth of the first ratchet gear may define a triangular space therebetween, and adjacent teeth of the plurality of teeth of the second ratchet gear may define a rectangular space therebetween,

In some aspects, the second ratchet gear may be fixed to the cam plate, such that the cam plate and the second ratchet gear rotate simultaneously with one another.

In further aspects, the cam plate may define a first spiral slot, and the proximal end portion of the first articulation shaft may have a protuberance received in the first spiral slot.

In other aspects, the hand-held surgical instrument may include a second articulation shaft having a protuberance extending from a proximal end portion thereof. The protuberance of the second articulation shaft may be received in a second spiral slot defined in the cam plate. The first and second articulation shafts may be configured to translate in opposite directions in response to a rotation of the cam plate.

In accordance with yet another aspect of the disclosure, a shaft assembly for use with a handle assembly of a hand-held surgical instrument is provided. The shaft assembly includes a knob housing, a shaft portion extending distally from the knob housing, an end effector coupled to a distal end portion of the shaft portion, an articulation lever rotationally coupled to the knob housing, a first articulation shaft, and a cam plate. The first articulation shaft is operably coupled to the articulation lever, such that a rotation of the articulation lever translates the first articulation shaft to articulate the end effector. The cam plate couples the articulation lever with a proximal end portion of the first articulation shaft. The cam plate is configured to urge the first articulation shaft in one of a proximal or distal direction in response to a rotation of the cam plate.

In aspects, the shaft assembly may further include an articulation locking assembly that includes a first ratchet gear operably coupled to the cam plate and a pawl engaged with the first ratchet gear. The pawl may be configured to restrict the rotation of the cam plate.

In some aspects, the first ratchet gear may be non-rotationally coupled to the articulation lever and pinned to the cam plate, such that a rotation of the articulation lever rotates the cam plate.

In further aspects, the cam plate may have a pin that extends through an elongate slot defined in the first ratchet gear. The first ratchet gear may be configured to rotate the cam plate after a delay.

In other aspects, the articulation locking assembly may include a second ratchet gear disposed between the first ratchet gear and the cam plate. The pawl may be engaged with the first and second ratchet gears.

In aspects, the first ratchet gear may have a plurality of teeth each defining an oblique surface, and the second ratchet gear may have a plurality of teeth each defining a linear surface.

In some aspects, adjacent teeth of the plurality of teeth of the first ratchet gear may define a triangular space therebetween, and adjacent teeth of the plurality of teeth of the second ratchet gear may define a rectangular space therebetween.

In other aspects, the second ratchet gear may be fixed to the cam plate, such that the cam plate and the second ratchet gear rotate simultaneously with one another.

In accordance with yet another aspect of the disclosure, a hand-held surgical instrument is provided and includes a handle housing, a shaft portion extending distally relative to the handle housing, a first articulation shaft supported in the shaft portion, a barrel cam, and an articulation motor. The first articulation shaft has a distal end portion configured to operably engage an end effector. The barrel cam is coupled to a proximal end portion of the first articulation shaft. The articulation motor may be operably coupled to the barrel cam and configured to rotate the barrel cam. A rotation of the barrel cam translates the first articulation shaft.

In aspects, the hand-held surgical instrument may further include a knob housing coupled to the handle housing. The knob housing may have the shaft portion extending distally therefrom. A manual rotation of the knob housing may rotate the shaft portion and the attached end effector.

In some aspects, the barrel cam may have an inner annular surface defining a helical cam slot. The proximal end portion of the first articulation shaft may have a protuberance received in the helical cam slot.

In further aspects, the hand-held surgical instrument may further include a ring gear non-rotationally coupled to the barrel cam. The articulation motor may have a motor gear operably coupled to the ring gear, such that a rotation of the motor gear results in a rotation of the barrel cam.

As used herein, the terms parallel and perpendicular are understood to include relative configurations that are substantially parallel and substantially perpendicular up to about +or −10 degrees from true parallel and true perpendicular.

### BRIEF DESCRIPTION OF THE DRAWINGS

Embodiments of the disclosure are described herein with reference to the accompanying drawings, wherein:

FIG. 1 is a perspective view of a hand-held electromechanical surgical instrument including a handle assembly, a shaft portion coupled to the handle assembly, and a surgical end effector coupled to the shaft portion, in accordance with an embodiment of the disclosure;

FIG. 2 is an enlarged side view, with a handle housing half removed, of the surgical instrument of FIG. 1;

FIG. 3 is a rear perspective view illustrating a handle portion of the surgical instrument in an open state for removing a battery;

FIG. 4 is a side cross-sectional view of the handle portion of FIG. 3, as taken through 4-4 of FIG. 3;

FIG. 5 is a longitudinal cross-sectional view, as taken through 5-5 of FIG. 1, illustrating a drive motor and a ball screw assembly for operating a stapling function of the end effector;

FIG. 6 is a perspective view, with parts separated, of a ball nut of the ball screw assembly of FIG. 5;

FIG. 7 is a side perspective view illustrating the shaft assembly of the surgical instrument of FIG. 1;

FIG. 8 is a perspective view, with parts separated, illustrating components of an articulation assembly of the shaft assembly of FIG. 7;

FIG. 9 is an enlarged perspective view, of the indicated area of detail of FIG. 8, illustrating first and second articulation shafts of the articulation assembly of FIG. 8;

FIG. 10 is a perspective view, with parts separated, illustrating a camming mechanism of the articulation assembly of FIG. 8;

FIG. 11 is a perspective view, with parts separated, illustrating an articulation locking assembly of the shaft assembly of FIG. 7;

FIG. 12 is a top view illustrating the articulation locking assembly of FIG. 11;

FIG. 13 is a side view illustrating another embodiment of a hand-held electromechanical surgical instrument including a handle assembly, a shaft portion coupled to the handle assembly, and a surgical end effector coupled to the shaft portion; and

FIG. 14 is a perspective view, with parts separated, illustrating components of an articulation assembly of the surgical instrument of FIG. 13.

### DETAILED DESCRIPTION

Embodiments of the presently disclosed surgical instruments are described in detail with reference to the drawings, in which like reference numerals designate identical or corresponding elements in each of the several views. As used herein the term “distal” refers to that portion of the surgical instrument, or component thereof, farther from the user, while the term “proximal” refers to that portion of the surgical instrument, or component thereof, closer to the user.

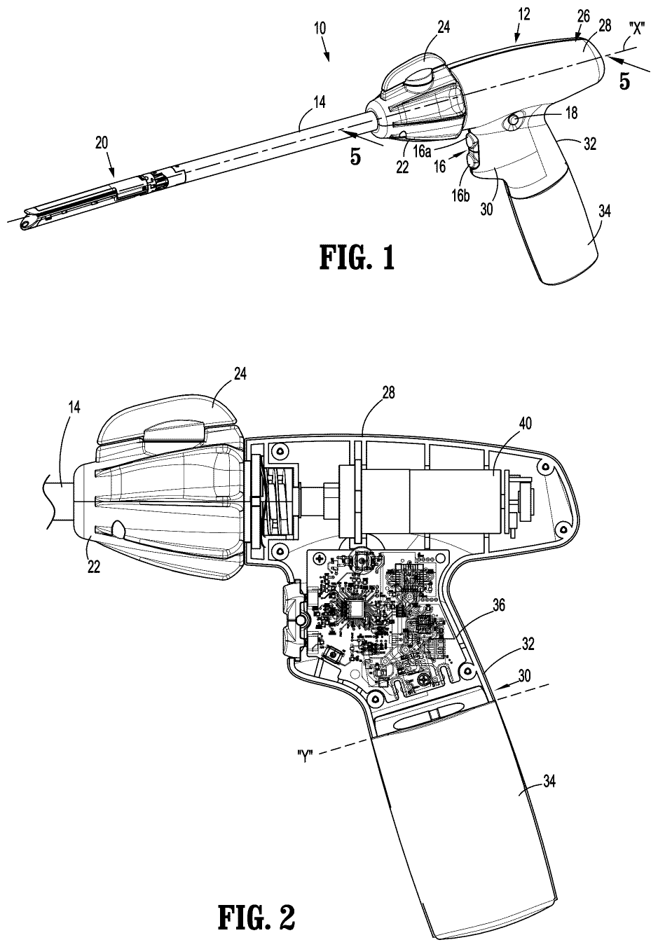

With reference to FIGS. 1 and 2, a surgical instrument, in accordance with an embodiment of the disclosure, is generally designated as 10, and is in the form of a powered hand-held electromechanical surgical instrument configured for selective coupling thereto of a plurality of different surgical end effectors, for example, the surgical end effector 20. The end effector 20 is configured for actuation and manipulation by the powered hand-held electromechanical surgical instrument 10.

The hand-held electromechanical surgical instrument 10 includes a handle assembly 12 and a shaft portion 14 extending distally from the handle assembly 12. The shaft portion 14 is configured for selective connection with a surgical attachment, such as, for example, the end effector 20. The handle assembly 12 has a fire switch 16 configured to actuate the various functions of the end effector 20. In addition, the handle assembly 12 has a safety switch 18 for preventing an inadvertent actuation of the fire switch 16. A knob housing 22 is rotationally coupled to the handle assembly 12 and configured to be manually rotated about a longitudinal axis “X” defined by the shaft portion 14 to rotate the end effector 20. An articulation lever 24 is rotationally coupled to the knob housing 22 and is configured to articulate the end effector 20 (e.g., move the end effector 200 along a horizontal plane between a position coaxial with the shaft portion 14 and multiple positions out of alignment with the shaft portion 14. The angular orientation of a longitudinal axis of the articulation lever relative to the longitudinal axis “X” corresponds to an angular orientation of a longitudinal axis of the end effector 20 relative to the longitudinal axis “X.” As such, the end effector 20 may articulate in the same direction and to the same angular extent as the articulation lever 24.

With reference to FIGS. 2-4, the handle assembly 12 includes a handle housing 26 consisting of a barrel portion 28 substantially aligned with the longitudinal axis “X,” and a handle portion 30 extending perpendicularly downward from the barrel portion 28. The handle portion 30 includes an upper segment 32 fixed to and extending downwardly from the barrel portion 28, and a lower segment 34 pivotably coupled to the upper segment 32. The handle assembly 12 includes a printed circuit board 36 supported in the upper segment 32 and a battery 38 disposed in the lower segment 34. The printed circuit board 36 is configured to be in electrical communication with the battery 38 and a drive motor 40. The motor 40 may be wirelessly connected, connected via a wire, or otherwise electrically connected to the printed circuit board 36 and the battery 38. The fire switch 16 may be a finger switch pivotably coupled to the upper segment 32 and has an upper button 16a and a lower button 16b each in communication with the printed circuit board 36 for activating the battery 38 to ultimately actuate an open/close and staple firing function of the end effector 20.

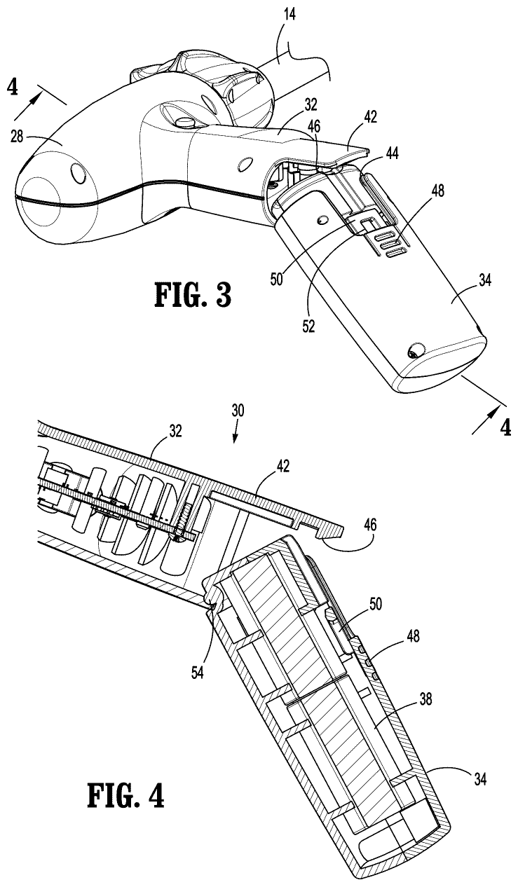

As shown in FIGS. 3 and 4, the upper segment 32 of the handle portion 30 has a flange 42, such as, for example, a tab, extending downwardly therefrom, and the lower segment 34 defines a cutout 44 configured for receipt of the flange 42. The flange 42 has an inner surface having a deflectable latch hook 46 at an end thereof, and the lower segment 34 includes a release button 48 having a tab 50 extending into the cutout 44. The tab 50 of the release button 48 defines a notch 52 configured to selectively receive and lock with the latch hook 46 of the upper segment 32. In some aspects, the upper and lower segments 32, 34 may detachably couple to one another via any suitable fastening connection, such as, for example, a bayonet-type connection. The upper and lower segments 32, 34 are pivotably coupled to one another about a hinge 54, such as, for example, a pivot pin. The handle portion 30 defines a plane that extends parallel with the central longitudinal axis “X” of the shaft portion 14, such that the lower segment 34 is pivotable relative to the upper segment 32 about a pivot axis “Y” (FIG. 2) that is parallel with the plane.

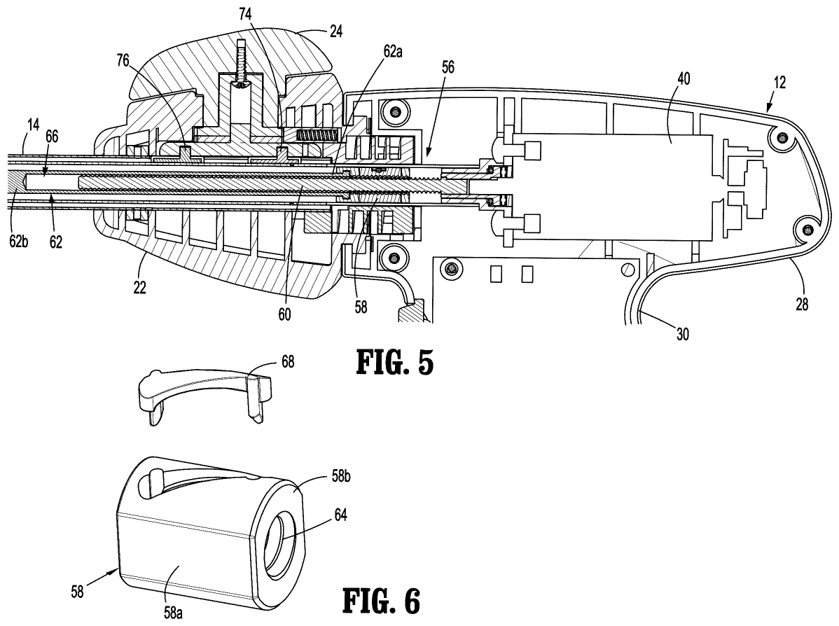

With reference to FIGS. 5 and 6, the surgical instrument 10 further includes a ball screw assembly 56 operably coupled to the motor 40 for carrying out an open/close and stapling function of the end effector 20 (FIG. 1). The ball screw assembly 56 includes a ball nut 58, a ball screw 60, and a firing shaft 62. The ball screw 60 is drivingly coupled to the motor 40 such that an actuation of the motor 40 results in a rotation of the ball screw 60. The ball nut 58 is non-rotationally supported in the shaft portion 14 and operably coupled to the ball screw 60. In particular, the ball nut 58 may have first and second planar lateral sides 58a, 58b that inhibit rotation of the ball nut 58 relative to the shaft portion 14, and the ball nut 58 has an inner surface defining threading 64 that houses bearings (not shown). The bearings are captured between the threading 64 of the ball nut 58 and a threaded outer surface of the ball screw 60.

The firing shaft 62 defines a conduit 66 through a proximal end portion 62a thereof. The ball screw 60 extends through the conduit 66 and the proximal end portion 62a is fixed to the ball nut 58. In this way, the firing shaft 62 moves with the ball nut 58 as the ball nut 58 moves axially within the shaft portion 14 and relative to the ball screw 60. The firing shaft 62 has a distal end portion 62b configured to operably couple to an axially-driven member (not shown) of the end effector 20. The ball nut 58 has a cap or cover 68 for containing the ball bearings therein.

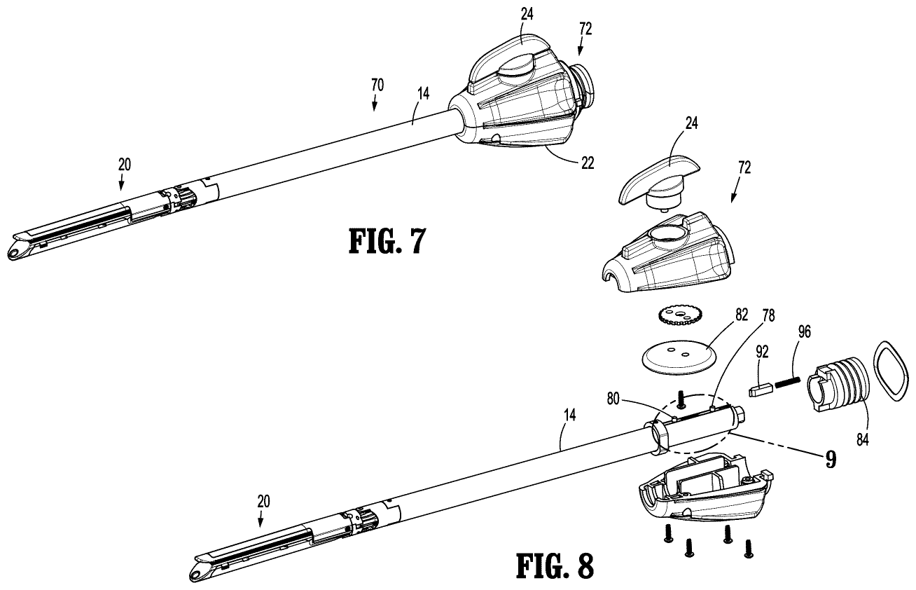

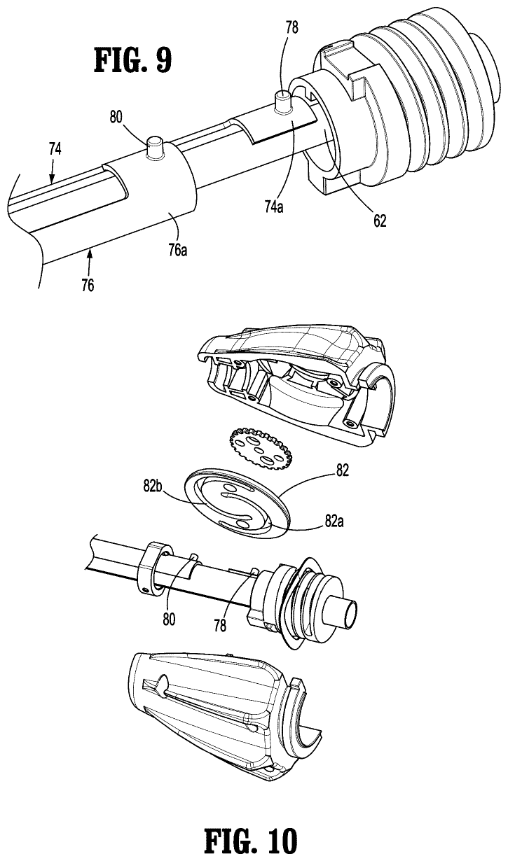

FIGS. 7-10 illustrate a shaft assembly 70 of the surgical instrument 10. The shaft assembly 70 includes the knob housing 22, the shaft portion 14, and the end effector 20. The knob housing 22 supports an articulation assembly 72 configured to effect the articulation of the end effector 20 relative to the shaft portion 14. The articulation lever 24 of the articulation assembly 72 is accessible from outside of the knob housing 22 and is configured to be manually rotated.

The articulation assembly 72 generally includes first and second articulation shafts 74, 76 and a cam plate 82. The first and second articulation shafts 74, 76 are axially movable within the shaft portion 14 and each has a proximal end portion 74a, 76a operably coupled to the articulation lever 22, and a distal end portion (not explicitly shown) operably coupled to opposite sides of the end effector 20. As such, a rotation of the articulation lever 22 translates the first and second articulation shafts 74, 76 in opposite directions to articulate the end effector 20. The proximal end portion 74a, 76a of each of the articulation shafts 74, 76 has a respective protuberance 78, 80. The cam plate 82 defines first and second spiral slots 82a, 82b for receiving the respective protuberances 78, 80. The spiral cam slots 82a, 82b are oriented so that a rotation of the cam plate 82 results in an axial movement of the first and second articulation shafts 74, 76 in opposite directions. A helical coil 84 may be attached to the proximal end portion of the shaft portion 14 for guiding an electrical cable (not shown) thereabout that runs from the motor 40 (FIG. 5). The helical coil 84 rotates with a rotation of the shaft portion 14 and prevents the cable from bunching and eliminates the need for an electrical slip ring.

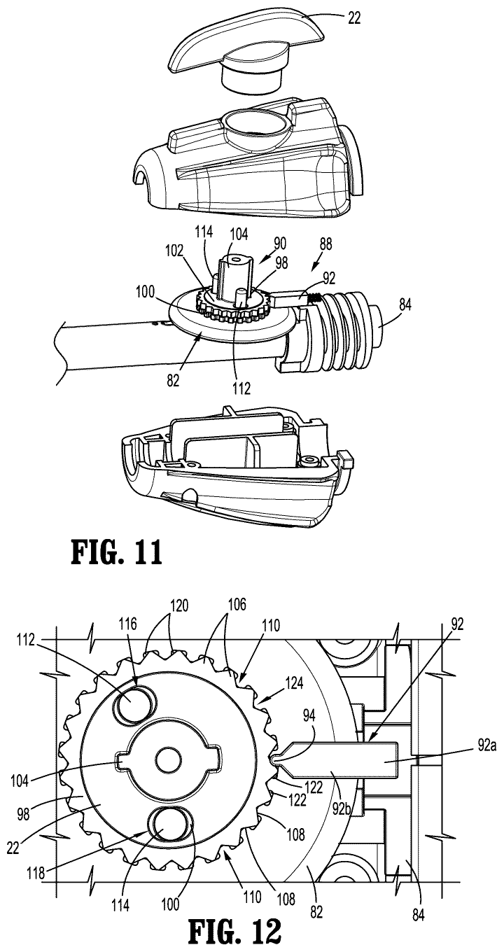

FIGS. 11 and 12 illustrate an articulation locking assembly 88 for selectively locking the articulation lever 22 in a rotational position to prevent backdriving of the articulation lever 22. The articulation locking assembly 88 generally includes a ratchet assembly 90 operably coupling the cam plate 82 and the articulation lever 22, and a pawl 92 engaged with the ratchet assembly 90 and configured to restrict the rotation of the cam plate 82. The pawl 92 has a proximal end portion 92a slidably supported on the spiral coil 84, and a free distal end portion 92b having an elongated distal tip 94. The pawl 92 is slidable along a longitudinal axis defined by the pawl 92. A detent spring 96 (FIG. 10) is provided to resiliently bias the distal tip 94 of the pawl 92 in a distal direction. In aspects, the pawl 92 may be resilient or rigid.

The ratchet assembly 90 includes a first ratchet gear 98 and a second ratchet gear 100. The first ratchet gear 98 has a plate 102 and a stem 104 extending from the plate 102. The stem 104 is received in a correspondingly shaped aperture (not explicitly shown) defined in the articulation lever 22 to non-rotationally couple the first ratchet gear 98 to the articulation lever 22. The plate 102 of the first ratchet gear 98 has a plurality of teeth 106 arranged around the outer periphery of the first ratchet gear 98. Each of the teeth 106 defines an oblique surface 108, such that adjacent teeth 106 define a triangular space 110 therebetween configured for selective receipt of the free distal tip 94 of the pawl 92.

The plate 102 of the first ratchet gear 98 is coupled to the cam plate 82, such that a rotation of the articulation lever 22 rotates the cam plate 82. For example, the cam plate 82 has a pair of pins 112, 114 that extend through a respective elongate slot 116, 118 defined in the first ratchet gear 98. The elongate slots 116, 118 define a length that is approximately 1.5 times greater than a diameter of the pins 112, 114 of the cam plate 82. In this way, a rotation of the first ratchet gear 98, in response to a rotation of the articulation lever 22, results in a rotation of the cam plate 82 after a delay.

The second ratchet gear 100 is disposed between the plate 102 of the first ratchet gear 98 and the cam plate 82. The second ratchet gear 100 is fixed to the cam plate 82 (e.g., via the pins 112, 114), such that the cam plate 82 and the second ratchet gear 100 rotate simultaneously with one another. The second ratchet gear 100 has a plurality of teeth 120 arranged around an outer periphery thereof. The teeth 120 of the second ratchet gear 100 each define a linear surface 122, such that adjacent teeth 120 of the second ratchet gear 100 define a rectangular space 124 therebetween configured for selective receipt of the distal tip 94 of the pawl 92. The distal tip 94 of the pawl 92 may be configured to wedge into the space 124 to resist rotation of the second ratchet gear 100 relative to the pawl 92.

The first and second ratchet gears 98, 100 are angularly oriented relative to one another so that the triangular spaces 110 of the first ratchet gear 98 overlap with the respective rectangular spaces 124 of the second ratchet gear 100. In aspects, the spaces 110 of the first ratchet gear 98 may assume the same shape as the spaces 124 of the second ratchet gear 100 and/or the spaces 110, 124 may assume any suitable shape, such as, for example, arcuate.

In operation, to articulate the end effector 20, the articulation lever 22 may be manually rotated in the direction intended for the end effector 20 to articulate. Rotation of the articulation lever 22 rotates the first ratchet gear 98, whereby one of the oblique surfaces 108 of the teeth 106 of the first ratchet gear 98 cams the free distal tip 94 of the pawl 92 proximally and out of the space 110 between the teeth 106 of the first ratchet gear 98 and the space 124 between the teeth 120 of the second ratchet gear 100. A tooth 106 of the first ratchet gear 98 is rotated into overlapping alignment with a space 124 defined between adjacent teeth 120 of the second ratchet gear 100, whereby the first ratchet gear 98 engages the pins 112, 114 of the cam plate 82 to drive a rotation of the cam plate 82. As described, rotation of the cam plate 82 translates the first and second articulation shafts 74, 76 in opposite directions. The opposing translation of the first and second articulation shafts 74, 76 drives the articulation of the end effector 20.

Due to the second ratchet gear 100 being fixed to the cam plate 82, the second ratchet gear 100 rotates with the cam plate 82 to maintain the teeth 106 of the first ratchet gear 98 in overlapping alignment with respective spaces 124 of the second ratchet gear 100. In this way, the distal tip 94 of the pawl 92 is maintained in a proximal position and out of the spaces 110, 124 of the ratchet gears 98, 100 while the articulation lever 22 is being rotated. However, upon removing the application of a rotational force on the articulation lever 22, the resilient bias of the pawl 92 (due to detent spring 96) will cam the first ratchet gear 98 to reposition the teeth 106 of the first ratchet gear 98 into overlapping alignment with the teeth 120 of the second ratchet gear 100. This is caused by the first ratchet gear 98 being free to rotate relative to the second ratchet gear 100 a selected distance. Despite any backdriving force exerted on the second ratchet gear 100 via the cam plate 82, rotation of the second ratchet gear 100 is resisted due to the engagement of the distal tip 94 of the pawl 92 in the space 124 of the second ratchet gear 100. More specifically, the adjacent teeth 120 of the second ratchet gear 100 capture the distal tip 94 of the pawl 92 therebetween, thereby resisting rotation of the second ratchet gear 100 and, in turn, the cam plate 82.

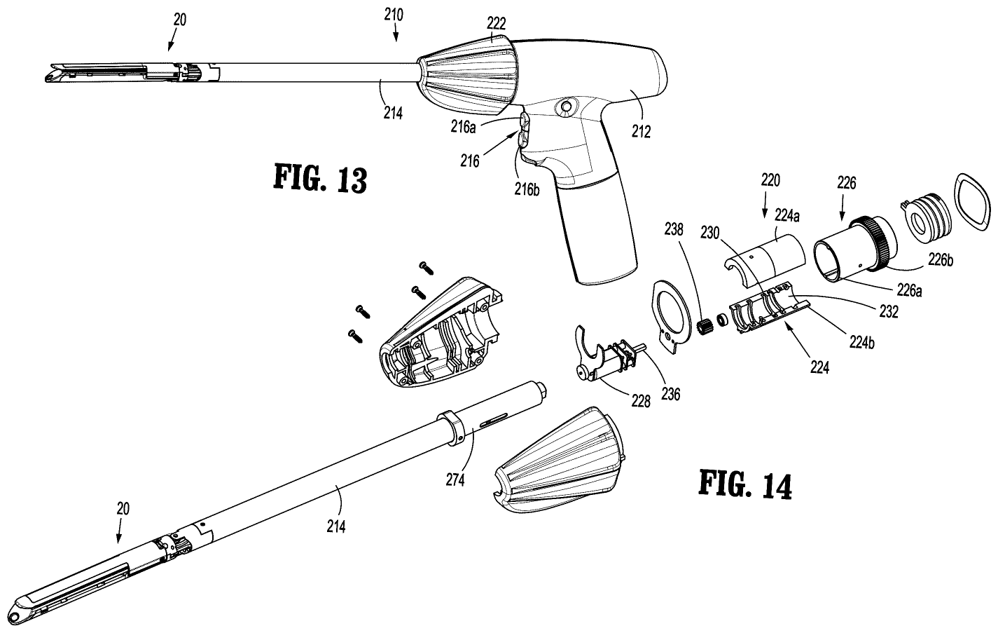

FIGS. 13 and 14 illustrate another embodiment of a hand-held surgical instrument 210, similar to the surgical instrument 10 described above. The surgical instrument 210 is different by having a powered articulation mechanism 220 rather than being manually actuated. Due to the similarities between the two surgical instruments, only those elements of the surgical instrument 210 deemed necessary to elucidate the differences from the surgical instrument 10 will be described in detail.

The surgical instrument 210 generally includes a handle housing 212, a knob housing 222 coupled to the handle housing 212, a shaft portion 214 extending distally from the knob housing 222, and an end effector, such as, for example, the end effector 20, operably coupled to a distal end portion of the shaft portion 214. An articulation switch 216 is pivotably coupled to the handle housing 212 for actuating an articulation of the end effector 20. The knob housing 222 may be manually rotated to thereby rotate the shaft portion 214 and the attached end effector 20 about a longitudinal axis defined by the shaft portion 214. The shaft portion 214 has a first articulation shaft 274 and a second articulation shaft (not explicitly shown) each supported therein.

The articulation mechanism 220 is received at least partially in the knob housing 222 and includes a barrel cam 224, a barrel cam gear 226, and an articulation motor 228. The barrel cam 224 consists of first and second semicircular half sections 224a, 224b together forming a tubular barrel cam 224. The barrel cam 224 is received within and fixed to the barrel cam gear 226. In some aspects, the barrel cam 224 may be monolithically formed with the barrel cam gear 226. Each of the first and second semicircular half sections 224a, 224b of the barrel cam 224 defines opposing helical cam slots 230 in an inner annular surface 232 thereof. The helical cam slots 230 receive a respective protuberance extending from the proximal end portion of the first and second articulation shafts 274. As such, a rotation of the barrel cam 224 results in axial translation of the first and second articulation shafts 274 in opposite directions.

The barrel cam gear 226 has a tubular body 226a and a ring gear 226b fixed about the tubular body 226a. The articulation motor 228 has a drive shaft 236 and a drive gear 238 non-rotationally coupled to the drive shaft 236. The drive gear 238 of the articulation motor 228 is operably coupled to the ring gear 226b to rotate the barrel cam 224 to translate the first and second articulation shafts 274.

In operation, an articulation switch 217 may be actuated to activate the articulation motor 228 to rotate the drive gear 238. Rotation of the drive gear 238 drives a rotation of the barrel cam 224 via the ring gear 226b. Due to the protuberances or pins of the articulation shafts 274 being received in the opposing helical cam slots 230 of the barrel cam 224, rotation of the barrel cam 224 drives an axial translation of the first and second articulation shafts 274 in opposing directions to articulate the end effector 20 relative to the shaft portion 214.

Any of the components described herein may be fabricated from either metals, plastics, resins, composites or the like taking into consideration strength, durability, wearability, weight, resistance to corrosion, case of manufacturing, cost of manufacturing, and the like.

It will be understood that various modifications may be made to the embodiments of the presently disclosed surgical instruments including switch assemblies. Therefore, the above description should not be construed as limiting, but merely as exemplifications of embodiments. Those skilled in the art will envision other modifications within the scope and spirit of the disclosure.

1. (canceled)

2. A hand-held surgical instrument, comprising:
a handle housing;
a shaft portion extending distally from the handle housing and defining a longitudinal axis, the shaft portion configured to couple to an end effector for treating tissue;
an articulation mechanism operably coupled to the shaft portion for transverse articulation of the end effector relative to the longitudinal axis; and
a rotatable barrel cam, comprising:
a first section having a first helical cam slot oriented in a clockwise direction and configured to engage a first articulation shaft of the articulation mechanism; and
a second section having a second helical cam slot oriented in a counterclockwise direction and configured to engage a second articulation shaft of the articulation mechanism;
wherein rotation of the barrel cam drives opposing longitudinal translation of the first articulation shaft and the second articulation shaft for articulating motion of the end effector.

3. The hand-held surgical instrument of claim 2, further comprising a motor operably coupled to the barrel cam for driving rotation thereof.

4. The hand-held surgical instrument of claim 3, further comprising a ring gear coupled to the barrel cam, and a drive gear coupled to the motor and engaged with the ring gear, wherein driving rotation of the ring gear leads to rotation of the barrel cam.

5. The hand-held surgical instrument of claim 3, further comprising a switch disposed on the handle housing and electrically coupled to the motor for actuating an articulation of the end effector.

6. The hand-held surgical instrument of claim 2, wherein the first helical cam slot is configured to receive a protuberance of the first articulation shaft for driving longitudinal translation of the first articulation shaft in a first direction.

7. The hand-held surgical instrument of claim 6, wherein the second helical cam slot is configured to receive a protuberance of the second articulation shaft for driving longitudinal translation of the second articulation shaft in a second direction opposite the first direction.

8. The hand-held surgical instrument of claim 2, further comprising a rotatable knob housing coupled to the handle housing.

9. The hand-held surgical instrument of claim 8, wherein the shaft portion is received at least partially within the knob housing.

10. The hand-held surgical instrument of claim 8, wherein the knob housing is manually rotatable for driving rotation of the shaft portion and the end effector about the longitudinal axis.

11. The hand-held surgical instrument of claim 8, wherein the articulation mechanism is received at least partially in the knob housing.

12. A shaft assembly for use with a handle assembly of a hand-held surgical instrument, the shaft assembly comprising:
a shaft portion defining a longitudinal axis and configured to couple to an end effector for treating tissue;
an articulation mechanism operably coupled to the shaft portion for transverse articulation of the end effector relative to the longitudinal axis; and
a rotatable barrel cam, comprising:
a first section having a first helical cam slot oriented in a clockwise direction and configured to engage a first articulation shaft of the articulation mechanism; and
a second section having a second helical cam slot oriented in a counterclockwise direction and configured to engage a second articulation shaft of the articulation mechanism;
wherein rotation of the barrel cam drives opposing longitudinal translation of the first articulation shaft and the second articulation shaft for articulating motion of the end effector.

13. The shaft assembly of claim 12, wherein the first section comprises a first semicircular half section with an inner surface having the first helical cam slot, and wherein the second section comprises a second semicircular half section with an inner surface having the second helical cam slot.

14. The shaft assembly of claim 12, further comprising a motor with a drive gear operably coupled to the barrel cam for rotation.

15. The shaft assembly of claim 14, wherein the barrel cam further comprises a ring gear operably coupled to the drive gear of the motor, such that the motor driving rotation of the ring gear leads to rotation of the barrel cam.

16. The shaft assembly of claim 14, further comprising a switch electrically coupled to the motor for actuating an articulation of the end effector.

17. The shaft assembly of claim 12, wherein the first articulation shaft comprises a projection receivable by the first helical cam slot for driving longitudinal translation of the first articulation shaft.

18. The shaft assembly of claim 12, further comprising a rotatable knob housing from which the shaft portion extends.

19. The shaft assembly of claim 18, wherein the shaft portion is received at least partially within the knob housing.

20. The shaft assembly of claim 18, wherein the knob housing is manually rotatable for driving rotation of the shaft portion and the end effector about the longitudinal axis.

21. The shaft assembly of claim 18, wherein the articulation mechanism is received at least partially in the knob housing.
